(19) United States
(12) Patent Application Publication
(10) Pub. No.: US 2026/0115149 A1
(43) Pub. Date: Apr. 30, 2026

(54) LIDOCAINE PATCH, AND METHODS OF MANUFACTURE AND USE

(71) Applicant: James Ciullo, Libertyville, IL (US)

(72) Inventor: James Ciullo, Libertyville, IL (US)

(21) Appl. No.: 19/370,984

(22) Filed: Oct. 28, 2025

Related U.S. Application Data
(60) Provisional application No. 63/712,779, filed on Oct. 28, 2024.

(51) Int. Cl. A61K 9/70; A61K 9/00; A61K 31/167; A61K 47/02; A61K 47/08; A61K 47/36

(52) U.S. Cl. CPC A61K 9/7053; A61K 9/0014; A61K 31/167; A61K 47/02; A61K 47/08; A61K 47/36

(57) ABSTRACT

A transdermal patch for delivering lidocaine for pain relief comprises a backing layer, an adhesive layer, and a composition containing lidocaine in an amount ranging from about 1% to about 10% by weight and heparin as a non-anticoagulant permeation enhancer in an amount ranging from about 1% to about 50% by weight. The heparin enhances permeation of lidocaine through the skin without exerting systemic anticoagulant effects by temporarily disrupting lipid bilayers in the stratum corneum to create microchannels for increased diffusion of lidocaine molecules. The patch provides effective relief for multiple pain types including neuropathic pain, inflammatory pain, ischemic pain, post-surgical pain, nociplastic pain, mechanical pain, and compressive pain. The transdermal patch is configured to provide sustained release of lidocaine for periods ranging from about 8 hours to about 24 hours, reducing pain by at least 50% with minimal systemic absorption.

### CROSS-REFERENCE TO RELATED APPLICATIONS

This application claims the benefit of U.S. Provisional Ser. No. 63/712,779 filed on Oct. 28, 2024, the entire disclosure of which is incorporated herein by reference.

### FIELD OF THE INVENTION

This invention is directed to a transdermal delivery patch comprising a local analgesic agent and a permeation enhancement agent, for reducing pain.

### BACKGROUND OF THE INVENTION

It is known that neuropathic pain including back pain, diabetic nerve pain, complex regional pain syndrome type II (CRPS-II), carpal tunnel syndrome, phantom limb pain, chemotherapy-induced neuropathy, or HIV sensory neuropathy and other nerve pain disorders have a predominantly neurological cause.

Presently, the symptoms of pain are predominantly treated pharmacologically with systemically active, oral or injectable analgesics and antiphlogistics, and, in part, in combination with psychosomatic or physical therapy, sometimes also in combination with other methods, such as, acupuncture. The last resort for diseases causing neuropathic pain, such as those of the intervertebral disk, is surgery.

An oral analgesic is carried into the patient's circulatory system and prevents the recognition of pain systemically by interrupting the transmission of pain signals from sensory neurons to the pain centers in the brain. Traditional oral analgesics include opioids (narcotics) such as morphine, codeine, methadone, Demerol® (meperidine hydrochloride) or Darvon® (propoxyphene hydrochloride); and non-steroidal anti-inflammatory drugs (NSAIDs), such as aspirin, ibuprofen or naproxen.

The systemic use of these drugs carries patient risk. Opioid use causes a variety of undesired side-effects, including sedation, dizziness, depression, nausea and constipation. Prolonged opioid usage carries a risk of patient addiction. The large and sometimes prolonged doses of non-steroidal anti-inflammatory drugs (“NSAIDs”) required to treat intense pain can cause gastric disorders, erosion of the stomach lining and intestinal mucus membrane, nephrotoxicity, hepatotoxicity, as well as internal bleeding. Orally administered drugs also cause side-affects that restrict physical activity (primarily in the case of opioids, due to sedation) and inhibit effective physical therapy.

In addition, neuropathic pain is often resistant to available drug therapies; a hallmark of neuropathic pain is its intractability. Typical non-steroidal anti-inflammatory drugs (NSAIDs) such as aspirin, indomethecin, and ibuprofen do not relieve neuropathic pain. The neuropathic pain observed in animal models predictive of human clinical outcome does not respond to NSAIDs.

Unfortunately, all systemic analgesics have a considerable number of undesirable side effects in common. The salicylic acid derivatives and nonsteroidal antiphlogistics are associated considerably and frequently with gastric disorders as a result of the antiproliferative active mechanism. Paracetamol, with a weaker effect, is associated with metabolic stress of liver and kidney functions, especially when used for a prolonged period of time and at required higher doses. Therefore, application of these therapies is limited by the spectrum of undesirable, product-specific effects in each case, because systemic interventions involve all of the organs and the organ systems. In addition, therapies such as surgery, in themselves present significant medical risks to the patient. These pharmacotherapies, do not represent sufficiently tolerable and effective forms of treatment.

Pain can also be treated locally by delivering a pain reliever directly to the site of pain or in a region near or surrounding the site of pain, through use of a local anesthetic.

Systemic application of local anesthetics might be applied invasively by means of injection. However, this option is practically eliminated due to the danger of systemic overdosage with, among others, serious cardiac side effects. Direct application of local anesthetics through local injection is technically possible and is performed in different ways. However, local injections are not only painful, but can also never be done directly by the patient. The local surface injection technique involves so-called neural therapy with muscular trigger points and requires experienced medical handling and technique (J. T. Travell, D. G. Simons, Myofascial Pain and Dysfunction, Vol. I/II, Williams & Wilkins, Baltimore, 1983). Therefore, this option is limited to use in clinically severe disorders. There is also the drawback that a local anesthetic injected into a highly vascularized area of the body can be carried away by the circulatory system and create the same risks as systemically administered anesthetics. This risk is increased when local anesthetic dosages are increased to manage intense pain. Further, use of conventional topical formulations, for example, creams, allows neither exact dosage nor continuous penetration over a prolonged period of application.

Microneedles (MNs) is a minimally invasive technology for transdermal drug delivery. These needles can painlessly penetrate the stratum corneum, allowing for the delivery of both small and large molecule drugs without causing discomfort. This approach improves patient compliance and opens up possibilities for self-administration.

Various types of microneedles, including solid, hollow, coated, and dissolving varieties. Each type has its own set of advantages and is suited for different applications. The choice of material for fabrication, ranging from silicon and metals to ceramics and polymers, significantly impacts the mechanical properties and drug delivery capabilities of the microneedles. However, the mechanical characterization of microneedles, including axial force, transverse force, and insertion force measurements, is crucial to ensure their ability to penetrate skin effectively without breaking, as mentioned in the reference titled “A Comprehensive Review of Microneedles: Types, Materials, Processes, Characterizations and Applications” by Faisal Khaled Aldawood et al. Polymers 2021, 13, 2815. and is incorporated in full by reference.

Other challenges of using microneedles-based drug delivery include difficulties in consistent skin penetration across different skin types, maintaining structural integrity during insertion, and ensuring drug stability within the microneedles. There can also be issues with scalability in manufacturing and patient discomfort, particularly in sensitive areas. Additionally, potential safety concerns such as infections from improper usage or inadequate sterilization can arise.

The topical administration of a local anesthetic overcomes some of the drawbacks of injection and microneedles. There is no need for the painfully invasive procedure and professional administration is not needed. The risk of the locally applied anesthetic acting as a systemically administered drug also is much reduced.

Dermal patches are well known to administer local anesthetics topically to patients at wound sites and to treat skin ailments. Dermal pain patches have a number of benefits, not the least of which is convenience. Amide and ester group-containing, for example, lidocaine of the amide type, exhibit, as a pharmacological active mechanism, an inhibition of the rapid sodium ion influx in nerve fibers. In this manner, the impulse conduction of the nerve path is blocked, which in principle involves all regional nerve fibers.

Generally, transdermal patches allow for continuous administration of medications through the skin. However, the skin's barrier properties pose a significant challenge in achieving effective drug permeation. The stratum corneum acts as a formidable barrier, significantly limiting the amount of drug that can pass through. As a result, many transdermal formulations require the use of permeation enhancers to facilitate the delivery of active pharmaceutical ingredients (APIs).

Transdermal patches bypass the need for needles, reducing the risk of complications such as infection, nerve damage, or bruising, which are common concerns with IV and injection methods. Patches allow for pain-free administration, improving patient comfort and reducing anxiety associated with needle use. The reference titled, “Recent Advancement of Medical Patch for Transdermal Drug Delivery,” Wong, W. F. et al., Medicina 2023, 59, 778, discloses non-invasive advantage of patches which make them more patient-friendly for chronic therapies and is incorporated in full by reference.

Unlike IV and injections, which deliver a high concentration of drugs quickly, leading to peaks and troughs in drug concentration, patches provide a controlled, steady release of the active ingredient. This helps maintain therapeutic levels of the drug over an extended period. The reference titled, “Enhancement strategies for transdermal drug delivery systems: current trends and applications,” published in PubMed Central of National Library of Medicine, highlights the sustained and controlled delivery offered by patches while reducing the need for frequent dosing seen in invasive methods.

Patches are convenient for self-administration without requiring healthcare personnel or specialized equipment. This makes them particularly useful for long-term therapies and chronic conditions where regular injections or IV infusions would be cumbersome. The reference titled, “Recent Advances in Transdermal Drug Delivery systems: a review” published in Biomaterials Research, discloses improved compliance and ease of use of patches compared to invasive techniques.

For instance, the lidocaine transdermal patch contains a high concentration of lidocaine—700 mg—but only about 35 mg effectively permeates through the skin. Hence, there is a need for effective permeation enhancers that can increase the amount of drug absorbed.

Typically, permeation enhancers modify the skin's barrier properties and making it more permeable to drugs. These enhancers can act through various mechanisms, such as disrupting the lipid bilayer of the stratum corneum, increasing skin hydration, or altering the physicochemical properties of the drug to improve solubility and diffusion.

Conventionally, the permeation enhancers enhance drug permeability while providing additional benefits, such as anti-inflammatory properties. However, some permeation enhancers are primarily recognized for its anticoagulant effects, which can complicate its use in transdermal formulations. The anticoagulant properties may pose risks, particularly in patients who may be prone to bleeding or have other contraindications.

Further, recent research has explored various non-anticoagulant enhancers, including fatty acids, such as lauric acid, that disrupt the lipid structure of the stratum corneum, surfactants that improve solubilization and penetration of drugs and chemical enhancers such as azone or menthol, which alter skin permeability without anticoagulant effects. Thus, there is a need for permeation enhancers that do not exert anticoagulant effects. Such enhancers enable the lidocaine patch to deliver significantly more amount of drug through the skin while potentially enhancing therapeutic efficacy.

### SUMMARY OF THE INVENTION

In one embodiment, it has surprisingly been found the present invention includes a transdermal patch for delivering lidocaine effectively by utilizing heparin as a permeation enhancer without its anticoagulant properties.

In another embodiment, the present invention intends to improve the permeation of lidocaine through the skin barrier, while ensuring higher drug absorption and therapeutic efficacy in localized pain management.

In yet another embodiment, the present invention provides a safe and non-invasive alternative to systemic drug delivery methods, such as injections or intravenous, while minimizing side effects.

In further embodiment, the present invention provides a formulation that maintains the stability of lidocaine and heparin within the patch, thereby optimizing both drug release and patient comfort.

In another embodiment, the present invention provides a transdermal patch that delivers lidocaine in concentrations ranging from about 1% to 10% by weight.

In yet another embodiment, the present invention enhances the permeation of lidocaine through the skin using heparin as a permeation enhancer in concentrations ranging from about 1% to 20% by weight, thereby optimizing drug delivery without anticoagulant effects.

In further embodiment, the present invention provides a formulation for a transdermal patch using adhesive materials like styrene-isoprene-styrene block copolymer for improved adhesion and comfort.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The teachings of all references cited herein are incorporated by reference in their entirety, regardless of whether they are specifically referred to or whether their teachings seem applicable to the present invention. This includes, but is not limited to, the structural, compositional, and procedural details described in each reference.

The present invention is directed to a patch for transdermal delivery of lidocaine for reducing pain, including neuropathic pain, osteoarthritis pain, back pain, pain from fibromyalgia, pain from muscle strains, pain from muscle sprains or bone degeneration pain or any combination thereof, comprising a composition comprising a therapeutically effective dosage of lidocaine and methods of use thereof.

The patch of the present invention may be used to reduce neuropathic pain, including back pain and/or discomfort. In addition, the patch of the present invention may be used to reduce osteoarthritis pain, pain from fibromyalgia, pain from muscle strains, pain from muscle sprains or bone degeneration pain or any combination thereof. The patch of the present invention may, in addition to a dosage of lidocaine, include treatment enhancing amounts of a permeation enhancement agent. The present invention may be used to reduce pain for extended time periods, e.g., 24 hours.

Before explaining at least one embodiment of the present invention in detail, it is to be understood that the invention is not limited in its application to the details of construction. The invention is capable of other embodiments or of being practiced or carried out in various ways. Also, it is to be understood that the phraseology and terminology employed herein are for the purpose of description and should not be regarded as limiting.

### I. Definitions

As used herein, the term “patch” refers to a medicated patch, e.g., a patch, comprising a composition comprising at least one active ingredient that is placed on the skin to deliver a continuous dosage of the active ingredient through the skin and into the surrounding tissue. In one embodiment, the active ingredient may penetrate deeply below the skin to a site of pain for deep tissue pain relief. In one embodiment, the active ingredient penetrates just below the skin to a site of pain localized therein for local pain relief. In one embodiment, the continuous dosage of the active ingredient provides minimal entry of the active ingredient into the blood stream. In another embodiment, the continuous dosage provides no entry of the active ingredient into the blood stream.

As used herein, the term “patch” may also be referred to herein as a “topical delivery system”, a “topical patch delivery system”, an “adhesive patch”, a “transdermal patch”, a “transdermal delivery system”, an “analgesic patch”, a “dressing”, a “topical carrier system”.

Transdermal patches are a well-accepted technology used to deliver a wide variety of pharmaceuticals. Patches of the present invention may be placed on the skin for specified therapeutic time periods and remain in place for up to 12 hours. Therapeutically effective dosages of pharmaceuticals are the choice for use with a patch of this invention.

Patches may comprise an adhesive to remain in place when placed on the skin or may be adhered by other means including adhesive tape or strips. In addition, patches of the present invention may be perforated and/or stretchable in order that they may be wrapped around an appendage or body part. In certain embodiments, a stretchable patch may be wrapped fully around an appendage or body part. In alternative embodiments, a stretchable patch may be wrapped partly around an appendage or body part. For example, a patch of the present invention may be wrapped around a knee, ankle, leg, elbow, wrist, finger, arm or neck. By wrapping the patch, pain relief may be provided at sites recalcitrant to a patch that would otherwise be expected to remain in place using just an adhesive, for example a moving joint such as an elbow, knee or wrist joint.

In one embodiment, the patch is an adhesive patch. In another embodiment, the patch is not adhesive. In yet another embodiment, the patch may be wrapped around a bodily appendage. In still another embodiment, the patch may be both adhesive and able to be wrapped around an appendage.

Conventional dermal patches include a carrier that holds a drug and allows the drug to be released onto a patient's skin for absorption. Many different kinds of dermal patches are known, including matrix type patches, reservoir-type patches, multi-laminate drug-in-adhesive type patches, matrix w/adhesive overlay and monolithic drug-in-adhesive type patches, and many others. Such patches can be readily prepared using technology which is known in the art such as described in Remington's Pharmaceutical Sciences, 18th 19th and 23rd editions, published by the Mack Publishing Company of Easton, Pa, and Remington Essentials of Pharmaceutics Edited by Linda Felton, Published by Pharmaceutical Press, ISBN 978 0 885711 105 0.

Patches of the present invention may include: (1) a backing layer, having an adhesive thereon; (2) an analgesic component for delivery of the analgesic, preferably, an analgesic in a carrier, referred to herein as an “analgesic composition” or “composition”; wherein the analgesic components are collectively referred to herein as the “active components” or “active ingredients”. These components are described in more detail below. In another embodiment, the analgesic composition comprises lidocaine.

Patches of the present invention can be any shape or size or can be customized to fit irregularly shaped body parts associated with pain, e.g., joints, back, neck, arms, legs, shoulders, hips, wrists, ankles, knees and/or fingers. For example, patches of the invention can be rectangular, square, round or oval in shape. Patches may also be perforated and stretchable for wrapping around different body appendages and/or joints, e.g., arms, legs, wrists, ankles, knees and/or fingers. Varying the size of the patch used varies the dosage. Often a patch is cut and only a portion is used. In some instances, the use of more than one patch may be advisable.

In one embodiment, patches are 10 cm×14 cm. In another embodiment, patches are smaller than 10 cm×14 cm. In yet another embodiment, patches are larger than 10 cm×14 cm.

In one embodiment, patches are cut to the size and shape needed for use in pain reduction. In one embodiment, the patch remains intact while the size of the patch changes upon stretching.

Patches suitable for use in the present invention include, but are not limited to, (1) the matrix patch; (2) the reservoir patch; (3) the multi-laminate drug-in-adhesive patch; and (4) the monolithic drug-in-adhesive patch; as described in Transdermal And Topical Drug Delivery Systems, pp. 249-297 (Tapash K. Ghosh et al. eds., 1997), and Dermal Drug Delivery, From Innovation to Production (edited by Tapash K. Ghosh, 2020) hereby incorporated in full herein by reference. These patches are well known in the art and generally available commercially.

The matrix patch comprises a drug containing matrix, an adhesive backing film overlay, and preferably, a release liner. In some cases, it may be necessary to include an impermeable layer to minimize drug migration into the backing film (e.g., U.S. Pat. No. 4,336,243, incorporated in full herein by reference). The drug-containing matrix is held against the skin by the adhesive overlay. Examples of suitable matrix materials include, but are not limited to, lipophilic polymers, such as polyvinyl chloride, polydimethylsiloxane, and hydrophilic polymers like polyvinylpyrrolidone, polyvinyl alcohol, hydrogels based on gelatin, or polyvinylpyrrolidone/polyethylene oxide mixtures.

The reservoir type patch design is characterized by a backing film coated with an adhesive and a reservoir compartment comprising a drug formulation, preferably in the form of a solution or suspension that is separated from the skin by a semipermeable membrane (e.g., U.S. Pat. No. 4,615,699, hereby incorporated in full herein by reference). The adhesive coated backing layer extends around the reservoir's boundaries to provide a concentric seal with the skin and hold the reservoir adjacent to the skin.

The monolithic/single drug-in-adhesive patch design is characterized by the inclusion of the drug formulation in the skin contacting adhesive layer, a backing film, and preferably, a release liner. The adhesive functions both to release the analgesic and adhere the analgesic matrix to the skin. The drug-in-adhesive system does not require an adhesive overlay and thus the patch size is minimized. Also, drug-in-adhesive type patches are thin and comfortable (e.g., U.S. Pat. No. 4,751,087, incorporated in full herein by reference).

The multi-laminate drug-in-adhesive patch design further incorporates an additional semi-permeable membrane between two distinct drug-in-adhesive layers or multiple drug-in-adhesive layers under a single backing film. See Peterson, T. A. and Dreyer, S. J. 21 Proceed. Intern. Symp. Control. Rel. Bioact. Mater. 477-478 (Nice, France 1994), hereby incorporated in full herein by reference).

The backing layer or backing serves as the upper surface of the patch and functions as the primary structural element and provides the patch with its flexibility. The material selected for the backing material should be selected so that it is substantially impermeable to the local analgesic and any other materials present; the backing is preferably made of a sheet or film of a flexible elastomeric material. The backing supports the active layers by way of an adhesive and holds the active layers against the application site. The combination of backing and adhesive should be biocompatible, non-irritating to the skin, breathable and able to hold the patch firmly against the skin.

Backings for use in patches of the invention are preferably made of a flexible, biocompatible material that imitates the elastic properties of skin and conforms to the skin during movement. Preferred have a moisture-vapor transmission rate similar to human skin. This reduces the chance of an infection developing under the patch after it is applied to a patient's skin.

Preferably, the backing layer is derived from synthetic polymers like polyolefin oils polyester, polyethylene, polyvinylidine chloride, and polyurethane or from natural materials like cotton, wool, etc. Non-occlusive backings allow the area to breathe (i.e., promote water vapor transmission from the skin surface). In one embodiment, the backing film is an occlusive polyolefin foil (Alevo, Dreieich, Germany). The polyolefin foil is preferably about 0.6 to about 1 mm thick. Other suitable backings are commercially available; for example, suitable backings can be purchased from 3M (St. Paul, Minn.) and Bertek (St. Albans, Vt.).

In one embodiment, the patch includes an occlusive dressing. In another embodiment, the patch includes a non-occlusive dressing. For example, a non-occlusive patch can enable moisture vapor on the surface of the skin to evaporate through the patch so as to prevent the undesired accumulation of moisture which, if it occurred, could cause the patch to fall off or even facilitate the growth of bacteria beneath the patch.

Permeable membranes can be used with patches of the present invention to overlay the portion of the patch adjacent to the skin to permit delivery of the patch's active ingredients to the application site. Preferably, the permeable membrane comprises a breathable material that is agreeable to the surface of a surgically closed wound and permits local delivery of local anesthetic into the skin of the patient at the wound site. Permeable membranes permit controlled delivery of the active components of the patch.

Permeable membranes useful in the present invention include thin non-porous ethylene vinyl acetate films or thin micro-porous films of polyethylene and polypropylene. Preferably, the permeable membrane is an ethyl vinyl acetate copolymer membrane. Suitable permeable membranes are commercially available; for example, suitable permeable membranes can be purchased from 3M (St. Paul, Minn.).

Adhesives may be used with patches of the present invention to adhere the active components to the backing and to adhere the backing to the patient's application site. Preferably, adhesives useful in the present invention can function under a wide range of conditions, such as, high and low humidity, bathing, sweating etc. Adhesives for use with patches of the present invention are well known in the art and selection is readily accomplished by an ordinary practitioner. Suitable adhesives include, but are not limited to, polyisobutylene-based adhesives, silicone-based adhesives, and acrylic-based adhesives. Preferably the adhesive is a composition based on natural or synthetic rubber; a polystyrene-based adhesive such as styrene-isoprene-styrene block copolymer, a polyacrylate such as, polybutylacrylate, polymethylacrylate, poly-2-ethylhexyl acrylate; polyvinylacetate; polydimethylsiloxane; and hydrogels (e.g., high molecular weight polyvinylpyrrolidone and oligomeric polyethylene oxide). Some of these adhesives are pressure-sensitive adhesives, which allow the patch to adhere to the skin with minimal pressure while being easy to remove without irritation.

Patches of the present invention deliver their medicine directly to the site of a person's pain. This may eliminate some of the side effects that come with oral dosing or local injections. For instance, some analgesics are likely to cause an upset stomach unless they're taken with food. And, because patches of the present invention provide minimal release of their active ingredient into the blood stream and provide release of their active ingredient slowly into the body tissues through the skin, people should also get more consistent pain relief than they do with oral dosing or injections.

The composition of the present invention is a pharmaceutical composition. The pharmaceutical composition of the invention includes pharmaceutically acceptable carriers.

As used herein, the terms “pharmaceutically acceptable carrier”, “carrier”, or “vehicle” refers to carrier materials suitable for transdermal drug administration. Carriers and vehicles useful herein include any such materials known in the art which are nontoxic and do not interact with other components. As used herein the term “a pharmaceutically acceptable carrier” refers to any substantially non-toxic carrier conventionally useable for transdermal administration of pharmaceuticals in which an active ingredient will remain stable and bioavailable. In one embodiment of the present invention, the local-analgesic of the composition of the present invention comprises a pharmaceutically acceptable carrier to contain and deliver the active component to the application site. As used herein, the term “carrier” may herein be interchangeable with the term “patch”

In certain embodiments, carriers are sterile and pharmaceutically acceptable for topical application and delivery of an active ingredient into or through a patient's skin. However, sterile compositions are more expensive to manufacturer such as requiring sterile packaging. Preferred functional characteristics of carriers are low adhesive strength, breathability, and conformability to the application area.

Pharmaceutically acceptable carriers for use in the invention are standard in the art, for example, matrix-type carriers, reservoir-type carriers, multi-laminate-type carriers, and monolithic drug-in-adhesive type carriers, such as those disclosed in “Transdermal And Topical Drug Delivery Systems” (Tapash K. Ghosh et al. eds., 1997); see also Kristine Knutson and Lynn K. Pershing, Topical Drugs, in Remington: The Science And Practice Of Pharmacy 866-885 (Alfonso R. Gennaro ed., 1995), see also Dermal Drug Delivery, From Innovation to Production(edited by Tapash K. Ghosh, 2020) the disclosures of which is hereby incorporated herein in full by reference.

Patches suitable for use in the present invention include, but are not limited to, (1) the matrix patch; (2) the reservoir patch; (3) the multi-laminate drug-in-adhesive patch; (4) matrix w/adhesive overlay and (5) the monolithic drug-in-adhesive patch; as described in Dermal Drug Delivery, From Innovation to Production, pp. 123-150 (edited by Tapash K. Ghosh, 2020), hereby incorporated in full herein by reference. These patches are well known in the art and generally available commercially.

In a preferred embodiment, the carrier is a matrix-type drug carrier. Matrix-type drug carriers are well known in the art. Suitable matrix-type drug carriers include, but are not limited to, the adhesives discussed below, such as polyisobutylene-based adhesives, silicone-based adhesives, and acrylic-based adhesives.

In another embodiment, the carrier is a hydrogel. Hydrogels are a mixture of water and a gelling agent, such as a hydrophilic polymer. In general, hydrogels form a three-dimensional lattice of polymer chains that retains an aqueous solution in a flexible, stable shape. Preferred hydrogels contain gelling agents distributed substantially uniformly throughout the carrier liquid, which is typically aqueous and may contain an alcohol and/or an oil.

Preferred gelling agents include, but are not limited to, crosslinked acrylic acid polymers such as carboxypolyalkylenes; hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methylcellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

Suitable hydrogels are commercially available, for example, suitable hydrogels can be purchased from BASF (St. Paul, Minn.) or Noveon (Cleveland, Ohio).

As used herein, the term “active ingredient” refers to a suitable drug that provides local analgesia or deep tissue analgesia, or a combination thereof, or a drug that provides a regional blockage of nervous pathways that carry pain signals. As used herein, the term “active ingredient”may also be referred to as “drug”or “active component”.

As used herein, the term “analgesia” refers to a neurological or pharmacological state characterized by an absence of normal sensibility to pain, without an effect on consciousness. Accordingly, painful stimuli are either not perceived at all, or they are moderated such that, even though they may still be perceived, they are no longer painful.

In one embodiment, an active ingredient may act as an analgesic. The analgesic may operate as a local analgesic and/or penetrate deeper and enter the blood stream. In one embodiment, the active ingredient functions as a local analgesic. In another embodiment, the active ingredient functions as an analgesic for deeper tissue. In yet another embodiment, the active ingredient does not enter the blood stream. In another embodiment, the active ingredient only minimally enters the blood stream.

In one embodiment, administration of a patch comprising a composition comprising the active ingredient, acts to reduce pain, including neuropathic pain, osteoarthritis pain, back pain, pain from fibromyalgia, pain from muscle strains, pain from muscle sprains, muscle contusions or bone degeneration pain or any combination thereof, in a subject.

As used herein, the term “reducing pain” refers to alleviating pain localized at a site of interest. The reduction of pain may include alleviating pain in an area around the site of interest.

As used herein, “pain,” includes both acute pain and chronic pain, which may be centralized pain, peripheral pain, or combination thereof.

As used herein, the term “acute pain” refers to centralized or peripheral pain that is intense, localized, sharp, or stinging, and/or dull, aching, diffuse, or burning in nature and that occurs for short periods of time.

As used herein, the term “chronic pain” refers to centralized or peripheral pain that is intense, localized, sharp, or stinging, and/or dull, aching, diffuse, or burning in nature and that occurs for extended periods of time (i.e., persistent and/or regularly reoccurring).

As used herein, the term “neuropathic pain” refers to any and all types of neuropathic pain regardless of the cause. Neuropathic pain refers to pain that originates from pathology of the nervous system. Neuropathic pain reflects both peripheral and central sensitization mechanisms.

Abnormal signals arise not only from injured axons but also from the intact nociceptors that share the innervation territory of the injured nerve. Neuropathic pain may result from lesions of the central nervous system, or from the peripheral nervous system. Neuropathic pain may also arise from disorders of ion channels, such as the sodium channels. The nervous system can generate and perpetuate pain (i.e., neuropathic), without any ongoing stimuli from injury. Neuropathic pain is often puzzling and frustrating for both patients and physicians because it seems to have no cause, responds poorly to standard pain therapies, can last indefinitely and even escalate over time, and often results in severe disability. The reduction of neuropathic pain as described herein refers to the alleviation or elimination of the neuropathic pain associated with a neuropathy.

Four pathological mechanisms are associated with the generation of pain in peripheral tissues in neuropathic pain conditions. These are: 1) nociceptor sensitization, whereby nociceptors have enhanced sensitivity to stimuli; 2) spontaneous activity related either to abnormal activity of transduction channels, or abnormal sensitivity of spike generation mechanisms; 3) abnormal coupling between sympathetic efferent fibers and nociceptors (sympathetically maintained pain); and 4) deafferentation, a central mechanism of pain whereby pain results from abnormal activity in neurons concerned with pain in the central nervous system as a result of altered input from primary afferents.

The primary sensory neurons that carry signals related to pain are called C-fiber and A-delta nociceptors. Normally, they fire action potentials in response to noxious mechanical, thermal, and/or chemical stimuli. Lumbar disk herniation with its accompanying chemical irritants to the adjacent nerve root can produce sciatic nerve pain. Carpal tunnel syndrome is due to a combination of repetitive stretching of the median nerve, compression caused by edema and hypertrophy of surrounding tissues, and inflammation producing chemical irritation of the median nerve. Trigeminal neuralgia has been attributed to vascular compression on the trigeminal nerve near the brain stem and may also relate to conditions such as multiple sclerosis.

Nerve fibers that have been damaged by injury or disease can fire spontaneously at the site of injury or at ectopic foci along the damaged nerve. Resulting paroxysms of pain are often described as lancinating, stabbing, or shooting. It is believed that when many nerve fibers are affected and fire asynchronously, neuropathic pain has a quality of continuous burning results. In addition, however, the nerve fibers that share the innervation territory of the injured nerve can also discharge abnormally. This discharge arises in the skin and therefore lends itself to topical therapy. Clonidine applied topically has been discovered to relieve pain after delivery to the painful site, for example.

Under normal conditions, sensations are transmitted from peripheral tissues via a connected chain of neurons in the spinal cord, brain stem, and brain. Interruption of any portion of that chain provides the potential for increased irritability and firing of nerves further up the pathway. This phenomenon explains how phantom limb pain can occur: Loss of sensory input from a limb can produce spontaneous firing of second-and third-order neurons, resulting in pain and other sensory experiences in the missing limb. Similarly, nerves damaged by diabetic neuropathy, post-herpetic neuropathy, or peripheral nerve trauma may generate firing in the higher-order nerves and, thus, ongoing pain.

Examples of specific sources of neuropathic pain for which the methods of the present invention can be used include autoimmune diseases, e.g., multiple sclerosis; metabolic diseases, e.g., diabetic neuropathies; back pain, spine or back surgery; postherpetic neuralgia; vascular disease; trauma; complex regional pain syndrome type II (CRPS-II); carpal tunnel syndrome; phantom limb pain; chemotherapy-induced neuropathy; central pain syndrome; trigeminal neuralgia; reflex sympathetic dystrophy syndrome; nerve compression; stroke; spinal cord injury; or HIV sensory neuropathy, or other nerve pain disorders having a predominantly neurological cause.

In contrast to feelings of immediate pain upon tissue injury, neuropathic pain can develop days or months after a traumatic injury. Furthermore, while pain caused by tissue injury is usually limited in duration to the period of tissue repair, neuropathic pain frequently is long lasting or chronic. Moreover, neuropathic pain can occur spontaneously or as a result of stimulation that normally is not painful sometimes called allodynia.

As used herein, the term “osteoarthritis pain” refers to pain associated with a degenerative joint disease where the cartilage that normally cushions the joint and protects it from impact erodes.

As used herein, the term “pain from fibromyalgia” refers to pain associated with a chronic condition characterized by diffuse or specific muscle, joint, or bone pain, along with fatigue and a range of other symptoms. Previously, fibromyalgia was known by other names such as fibrositis, chronic muscle pain syndrome, psychogenic rheumatism and tension myalgias.

As used herein, the term “back pain” refers to pain associated with all regions of the back including lower, mid and upper back pain and includes pain from a wide range of causes, which include but are not limited to mechanical, degenerative, inflammatory, neuropathic, and lifestyle-related factors. Mechanical Causes—Herniated or bulging discs: When discs in the spine become compressed or ruptured, they can press on nearby nerves, causing pain that can radiate to other areas, such as down the leg (sciatica) if the lower back is affected, and spondylolisthesis: This condition occurs when a vertebra slips forward over the one below; Degenerative causes include degenerative disc disease: Natural aging can cause discs to lose moisture and elasticity, reducing their ability to absorb shocks. This degeneration can lead to bone-on-bone contact and inflammation, causing chronic back pain, and spinal stenosis: Narrowing of the spinal canal, often due to arthritis, causes pressure on the spinal cord and nerves, resulting in pain, numbness, and weakness in the back and limbs; Inflammatory causes including but not limited to ankylosing spondylitis, sacroiliitis, autoimmune disorders; Neuropathic Causes include but are not limited to Sciatica: Often caused by herniated discs or spinal stenosis, sciatica is characterized by pain that radiates from the lower back down one leg. This pain results from irritation or compression of the sciatic nerve, Nerve Entrapment: Nerves can become trapped or compressed due to structural changes in the spine or surrounding muscles, leading to sharp, shooting pain or a burning sensation, often in a specific area, Shingles: Shingles, a viral infection caused by the varicella-zoster virus, can cause severe neuropathic pain along the affected nerve pathways, including the back, Obesity: Excess body weight puts additional strain on the spine, particularly the lower back, increasing the risk of conditions like disc degeneration and joint strain, Smoking: Smoking is associated with decreased blood flow to the spine, accelerating degenerative changes and increasing the risk of osteoporosis and disc disease, Other medical conditions including but not limited to osteoporosis, kidney disease, fibromyalgia, obesity, stress and mental health: Emotional stress, anxiety, and depression can contribute to back pain by causing muscle tension or worsening the perception of pain. Chronic stress can also lead to poor posture and muscle tightness, creating a cycle of pain.

As used herein, the term “bone degeneration pain” refers to pain associated with conditions leading to degenerative bone disorders characterized by low bone mass and microarchitectural deterioration of bone tissue, leading to enhanced bone fragility and increased fracture risk. Also, as used herein, the term “bone pain” refers to pain associated with conditions leading to bone breaks, fractures, contusions, bruises and any other bone deformity. Specific bones effected are disclosed in The Complete Human Body: The Definitive Visual Guide (DK Human Body Guides) (ISBN-13: 9780744073676 and ISBN-10: 0744073677), Dr. Alice Roberts 3rd edition 2023, which is fully incorporated by reference.

As used herein, the term “pain from muscle strains” refers to pain associated with muscle tears and/or pulled muscles. Muscle strains occur when an excessive amount of force or pressure is directed onto muscles that cause damage or tearing to the muscle fibers and/or surrounding tendons. common muscle strains, torn muscles and pulled muscles are: Achilles tendon tear, pulled backs, lower back muscle strain, tearing the rotator cuff, abs (abdominal) muscle strains, calf muscle strain, hamstring muscle strain, quads (quadriceps) muscle strain, leg muscle strain, knee (or plantaris) muscle strain, chest muscle strain, groin pull or muscle strain, bicep muscle strain, and arm muscle strain. Other muscles that can be strained, torn and pulled are disclose in The Complete Human Body: The Definitive Visual Guide (DK Human Body Guides) (ISBN-13: 9780744073676 and ISBN-10: 0744073677), Dr. Alice Roberts 3rd edition 2023, which is fully incorporated by reference

As used herein, the term “pain from muscle sprains” refers to pain associated with a stretch or tear of a ligament, the band of connective tissues that joins the end of one bone with another. Sprains are caused by trauma such as a fall or blow to the body that knocks a joint out of position and, in the worst case, ruptures the supporting ligaments. Common muscles impacted by sprains are disclosed in The Complete Human Body: The Definitive Visual Guide (DK Human Body Guides) (ISBN-13: 9780744073676 and ISBN-10: 0744073677), Dr. Alice Roberts 3rd edition 2023, which is fully incorporated by reference.

As used herein, the term “pain from muscle contusion” refers to pain when there is a direct impact on the muscle, typically from a blunt force, such as a fall or a blow. This trauma causes damage to the muscle fibers and underlying blood vessels, leading to bleeding under the skin, which creates visible bruising. Pain, swelling, and limited movement often accompany a contusion as blood collects in the area. Common muscles impacted by contusions are disclosed in The Complete Human Body: The Definitive Visual Guide (DK Human Body Guides) (ISBN-13: 9780744073676 and ISBN-10: 0744073677), Dr. Alice Roberts 3rd edition 2023, which is fully incorporated by reference.

The patches of the present invention can be used to reduce pain such as neuropathic pain. For example, the patches of the present invention may be used to reduce pain associated with diabetic neuropathy, back pain, carpel tunnel syndrome or other pains associated with nerve injury or any combination thereof. In addition, the patches of the present invention may also be used to reduce pain associated with fibromyalgia, muscle strains, muscle sprains, muscle contusions osteoarthritis or bone degeneration or any combination thereof.

Compositions of this invention are described below. In some embodiments, any of the compositions of this invention will comprise lidocaine, in any form or embodiment as described herein. In some embodiments, any of the compositions of this invention will comprise a combination of lidocaine and menthol, in any form or embodiment as described herein. In some embodiments, any of the compositions of this invention will consist of lidocaine, in any form or embodiment as described herein. In some embodiments, any of the compositions of this invention will consist of a combination of lidocaine and menthol, in any form or embodiment as described herein. In some embodiments, any of the compositions of this invention will consist essentially of lidocaine, in any form or embodiment as described herein. In some embodiments, any of the compositions of this invention will consist essentially of a combination of lidocaine and menthol, in any form or embodiment as described herein. The term “comprise” refers to the inclusion of the indicated active agents, such as a combination of lidocaine and menthol, as well as inclusion of other active agents, and pharmaceutically acceptable carriers, excipients, emollients, stabilizers, etc., as are known in the pharmaceutical industry. The term “consisting essentially of” refers to a composition, whose only active ingredients are the indicated active ingredients, however, other compounds may be included which are for stabilizing, preserving, etc. the formulation, but are not involved directly in the therapeutic effect of the indicated active ingredients. The term “consisting essentially of” may refer to components which facilitate the release of the active ingredients. The term “consisting” refers to a composition, which contains the active ingredient and a pharmaceutically acceptable carrier or excipient.

The lidocaine patch is commonly used to treat a number of pain types. It provides localized pain relief by delivering lidocaine directly to the affected area, effectively managing both acute and chronic pains. The includes nociceptive pain such as superficial and deep somatic pain, inflammatory pain associated with conditions like arthritis, as well as neuropathic pain types including diabetic neuropathy, postherpetic neuralgia, and focal neuropathy. Additionally, the lidocaine patch is used for managing mixed pain conditions, like complex regional pain syndrome (CRPS), and for certain cancer-related pains, such as nerve compression pain.

Lidocaine patches can also alleviate conditions associated with functional pain syndromes, breakthrough pain, and refractory pain, especially when other treatments have proven ineffective. With its versatility, the lidocaine patch is an essential tool for addressing various pains such as superficial nociceptive pain, referred pain (e.g., angina-related pain), and even some specific and challenging conditions like phantom limb pain, allodynia, and hyperalgesia.

A detailed list of pains which the lidocaine patch can treat is distinguished by duration, origin, mechanism, location, intensity, specific syndromes, and other unique characteristics below:

By duration, pain can be acute or chronic. Acute pain is short-term and linked to specific injuries, while chronic pain persists and can be classified further into chronic primary pain or chronic secondary pain, which may relate to an underlying condition.

When classified by origin, pain is divided into nociceptive, neuropathic, and mixed categories. Nociceptive pain is typically somatic or visceral. Somatic pain can be superficial (cutaneous) or deep, affecting structures like muscles and bones. Visceral pain originates in internal organs, presenting in various forms such as appendicitis pain, renal pain, biliary pain, parturition pain, or referred visceral pain, where pain is felt in an area distinct from the source. Neuropathic pain results from nerve damage and is either peripheral or central. Peripheral neuropathic pain includes conditions like diabetic neuropathy, postherpetic neuralgia, carpal tunnel syndrome, chemotherapy-induced neuropathy, trigeminal neuralgia, and focal neuropathy. Central neuropathic pain involves multiple sclerosis, post-stroke pain, spinal cord injury pain, and phantom limb pain. Mixed pain combines nociceptive and neuropathic elements, often presenting in conditions like complex regional pain syndrome (CRPS) or chronic postsurgical pain.

Mechanistic classifications identify inflammatory pain, mechanical pain, and ischemic pain. These distinctions help define the underlying processes responsible for pain sensations.

Pain can also be categorized by its location, with superficial pain affecting skin and tissues close to the surface and deep pain involving structures like joints and tendons.

Intensity-based classification identifies pain as mild, moderate, severe, or excruciating. Specific pain syndromes encompass categories like cancer pain including tumor-related pain, bone pain, chemotherapy- and radiation-induced pain, and nerve compression pain, fibromyalgia pain, headache disorders including tension headaches, migraines, and cluster headaches, arthritis pain (osteoarthritis and rheumatoid arthritis), and back pain (mechanical and radicular types). CRPS is a complex syndrome subdivided into Type I and Type II, both involving chronic and often debilitating pain. Other notable conditions include trigeminal neuralgia and postherpetic neuralgia, both of which result in severe nerve pain.

Miscellaneous pain types include psychogenic pain, linked to emotional or psychological distress; refractory pain, which is resistant to standard treatments; and conditions like allodynia and hyperalgesia, where there is an exaggerated response to stimuli that may not normally cause pain. Breakthrough pain refers to intense episodes that temporarily exacerbate chronic pain, and incident pain is related to specific movements or activities. End-of-dose failure pain occurs when pain resurfaces as medication effectiveness diminishes before the next scheduled dose.

Additionally, pain classifications can include nociceptive types such as somatic and visceral pain, and inflammatory pain including post-injury, postoperative, or autoimmune-related, as well as pain from conditions like arthritis. Functional pain syndromes include fibromyalgia, irritable bowel syndrome, tension-type headaches, and noncardiac chest pain. Cancer-related pain can encompass tumor-related pain, bone pain, and pain induced by treatments like chemotherapy or radiation. Breakthrough pain is either cancer-related or non-cancer related.

Further classifications include mixed pain, CRPS, referred pain including angina and gallbladder pain, phantom pain including phantom limb pain, allodynia, and hyperalgesia. Chronic conditions causing neuropathic pain range widely from abdominal wall defect to conditions like diabetes, Guillain-Barré syndrome, various forms of arthritis such as rheumatoid, psoriatic, ankylosing spondylitis, gout, lupus, juvenile idiopathic, and reactive; demyelinating diseases, and complex regional pain syndromes (CRPS), among many others which can be found in International Association for the Study of Pain (IASP) definitions of pain 2011 edition and Wall and Melzack's Textbook of Pain by McMahon et al., 6th edition, Pain A textbook for Health Professionals by Hubert van Griensven, 2nd edition 2013 ISBN 13: 9780702034787 and 3rd edition 2023 ISBN 13: 978-0323870337 all of which are incorporated in full by reference.

The lidocaine patch of the present invention is also effective for treating inflammatory pain, which results from immune cell activation leading to release of cytokines, prostaglandins, bradykinin, and histamine, causing nociceptor sensitization and peripheral or central sensitization through neuroimmune cross-talk. Inflammatory pain is characterized by key features including warmth, redness when superficial, swelling, throbbing or aching sensations, tenderness, pain that worsens with movement, and responsiveness to anti-inflammatory medications such as NSAIDs and steroids, and may produce systemic signs including fever and raised CRP or ESR levels. Major subtypes of inflammatory pain treatable by the lidocaine patch include acute inflammatory pain such as infectious conditions including cellulitis, abscess, septic arthritis, and osteomyelitis, traumatic injuries including sprains and strains with local inflammation, and post-operative inflammation during the early zero to two week period. Chronic inflammatory pain conditions treatable by the patch include autoimmune rheumatic diseases such as rheumatoid arthritis involving synovitis and pannus formation, psoriatic arthritis, and ankylosing spondylitis, chronic inflammatory bowel disease including Crohn's disease and ulcerative colitis causing abdominal and visceral pain, chronic prostatitis and chronic pelvic inflammatory pain whether infectious or noninfectious in origin, and autoimmune neuropathies with inflammatory etiology such as vasculitic neuropathy.

Additional inflammatory pain conditions treatable by the lidocaine patch include inflammatory visceral pain from conditions such as pancreatitis, appendicitis, cholecystitis, and peritonitis, which present as deep, poorly localized pain often accompanied by autonomic signs including nausea and diaphoresis, inflammatory bone or periosteal pain from conditions including osteomyelitis, osteitis condensans, and sickle cell vaso-occlusive crises which involve mixed ischemic and inflammatory mechanisms, and neuroinflammatory pain resulting from cytokine-mediated neuropathic pain associated with conditions including HIV, certain autoimmune diseases, and CAR-T syndrome, as well as microglial activation in the central nervous system leading to central sensitization in chronic inflammatory states. Inflammatory pain can be further classified by driver mechanism including infective causes such as bacterial, viral, or fungal infections, autoimmune or autoinflammatory causes involving immune system attack, sterile inflammatory causes such as crystal arthropathies including gout and pseudogout, and drug-induced inflammatory conditions such as immune checkpoint inhibitor arthralgia and serum sickness. Typical diagnostic clues for inflammatory pain include local signs of inflammation, elevated inflammatory markers such as CRP and ESR, synovial fluid analysis findings, imaging showing synovial enhancement, and biopsy when needed. Common pain qualities associated with inflammatory pain include aching, throbbing, pressure sensations, pain aggravated by movement, and pain partially relieved by anti-inflammatory medications.

Examples of inflammatory pain by organ system treatable by the lidocaine patch include musculoskeletal inflammatory pain such as rheumatoid arthritis, gout, tendonitis, bursitis, and polymyalgia rheumatica, skin and subcutaneous inflammatory pain including cellulitis and erysipelas, visceral inflammatory pain such as appendicitis and cholecystitis, and vasculitis-related pain including giant cell arteritis presenting with headache and jaw claudication, and cutaneous vasculitis presenting with tender palpable purpura. The lidocaine patch is also effective for ischemic pain, which occurs due to inadequate tissue perfusion leading to anaerobic metabolism, local acidosis, and release of algogenic metabolites such as adenosine and bradykinin that activate nociceptors, sometimes combined with neuropathic changes after prolonged ischemia. Ischemic pain is characterized by severe intensity, often presenting as sharp, pressure-like or cramping sensations, worsening with activity or exertion in conditions such as claudication, or occurring continuously in conditions such as critical ischemia, and may be accompanied by physical signs including pallor, coolness, pulselessness, paresthesia, and motor weakness.

Major subtypes of ischemic pain treatable by the lidocaine patch include cardiac ischemic pain such as angina pectoris in both stable and unstable forms and myocardial infarction presenting as pressure or heavy sensations radiating to the jaw or arm, limb ischemia including intermittent claudication from peripheral arterial disease and acute limb ischemia with severe rest pain and risk of tissue loss, visceral ischemia such as mesenteric ischemia with postprandial pain and weight loss, renal ischemia with flank pain, and intestinal ischemia with severe abdominal pain out of proportion to physical examination findings, ischemic neuropathic pain including ischemic mononeuropathy and vasculitic ischemic neuropathy, sickle cell vaso-occlusive pain presenting as bone pain crises involving mixed ischemic and inflammatory mechanisms with severe deep aching, and ischemic fasciitis or compartment syndrome presenting with excruciating pain, tense swollen compartments, and pain with passive stretch. Typical diagnostic clues for ischemic pain include vascular studies such as Doppler ultrasound, ankle-brachial index measurements, CT or MR angiography, ECG and troponin testing for cardiac ischemia, and lactate levels and CT imaging for mesenteric ischemia. Pain qualities and patterns associated with ischemic pain include crushing or pressure sensations in cardiac ischemia, cramping in claudication, severe unrelenting pain in acute ischemia, and pain disproportionate to physical findings in mesenteric ischemia. Many ischemic pain conditions represent medical emergencies including myocardial infarction, acute limb ischemia, mesenteric ischemia, and compartment syndrome.

Post-surgical pain is another category effectively treated by the lidocaine patch, which can be nociceptive in nature from incision or tissue injury, neuropathic from nerve transection or compression, inflammatory from wound response, ischemic from flap ischemia, or mixed in nature, with risk for chronic post-surgical pain if persistent beyond normal healing. Post-surgical pain is timeline dependent and includes acute immediate postoperative pain, subacute healing pain, and chronic post-surgical pain persisting beyond three months, and may include clinical features such as allodynia, hyperalgesia, neuropathic descriptors including burning and electric sensations, and function loss. Subtypes of post-surgical pain by timeline include immediate or acute pain lasting hours to weeks from incision pain, visceral manipulation pain, and procedure-related nerve irritation, subacute pain lasting weeks to three months from wound healing, seroma or hematoma pain, and early neuropathic signs, and chronic post-surgical pain defined as pain persisting longer than three months that is not explained by other causes such as infection or recurrence.

Common etiologies and examples of post-surgical pain treatable by the lidocaine patch include nerve injury or transection resulting in post-mastectomy pain syndrome, post-hernia repair ilioinguinal neuralgia, and neuropathic pain after limb amputation including phantom pain and neuroma, entrapment or compression from scar tissue or implants such as mesh-related pudendal or groin pain, visceral adhesions causing chronic abdominal pain after laparotomy, post-thoracotomy pain syndrome with chronic intercostal neuralgia, neuroma formation producing sharp lancinating pain at the stump, ischemic flap or graft pain from vascular compromise after reconstruction, and device-related pain such as painful spinal cord stimulator lead migration and infected implant pain. Risk factors for chronic post-surgical pain include pre-existing chronic pain, high acute postoperative pain intensity, young age, female sex, psychological factors including anxiety and catastrophizing, and certain surgeries including amputation, thoracotomy, mastectomy, inguinal hernia repair, breast surgery, and spinal surgery. Typical pain descriptors for post-surgical pain include incisional burning or dull ache, neuropathic electric or stabbing sensations, activity-related exacerbation, allodynia around scars, and painful range of motion limitations from adhesions. Prevention and treatment approaches for post-surgical pain include multimodal analgesia perioperatively, regional anesthesia with nerve blocks, minimizing nerve transection during surgery, early mobilization, and use of neuropathic agents for persistent neuropathic chronic post-surgical pain.

Nociplastic pain, also referred to as centralized pain, represents altered central nociceptive processing involving central sensitization, dysfunctional descending inhibition, augmented facilitatory pathways, and neuroimmune and neuroendocrine contributors without clear ongoing peripheral tissue damage or nerve lesion sufficient to explain the pain. The lidocaine patch may provide relief for nociplastic pain conditions characterized by widespread pain, pain disproportionate to observable injury, high comorbidity of fatigue, sleep disturbance, mood disorders, cognitive symptoms, pain hypersensitivity including allodynia and hyperalgesia, and tender points, with variable response to classic analgesics but often benefiting from neuromodulatory approaches, behavioral interventions, exercise programs, and centrally-acting medications. Core nociplastic pain conditions treatable by the lidocaine patch include fibromyalgia characterized by widespread musculoskeletal pain, fatigue, and cognitive dysfunction known as fibro fog, chronic widespread pain syndromes overlapping with the fibromyalgia spectrum, irritable bowel syndrome involving visceral nociplastic pain, temporomandibular disorders with orofacial nociplastic pain, chronic tension-type headache with central features, chronic pelvic pain syndromes with overlapping nociplastic features including endometriosis which sometimes presents with mixed mechanisms, interstitial cystitis or bladder pain syndrome, non-cardiac chest pain from esophageal hypersensitivity, and post-COVID chronic pain syndromes that appear to be centralized in many patients.

The pathophysiology of nociplastic pain involves multiple mechanisms including central sensitization characterized by increased dorsal horn excitability, NMDA receptor activation, and wind-up phenomena, impaired descending inhibition with reduced conditioned pain modulation, neuroplastic changes including cortical map reorganization and altered resting-state networks, neuroimmune mechanisms with microglial activation that can maintain central sensitization, and neuroendocrine dysregulation including HPA axis dysregulation and autonomic dysfunction. The clinical profile of nociplastic pain includes widespread migratory pain, disproportionate functional impairment, sleep disturbance, mood and cognitive complaints, sensory testing that may show widespread hyperalgesia, and routine laboratory tests and imaging that are often unremarkable although such testing should be used to rule out other causes. Management principles for nociplastic pain include a multimodal multidisciplinary approach incorporating graded exercise therapy, cognitive behavioral therapy and pain psychology interventions, sleep optimization, low-dose serotonin-norepinephrine reuptake inhibitors or tricyclic antidepressants, gabapentinoids with variable effectiveness, nonpharmacologic therapies including exercise, acupuncture, and mindfulness practices, and social and vocational rehabilitation.

Additional pain categories treatable by the lidocaine patch include mechanical or compressive pain resulting from physical compression or overload such as disc herniation, facet arthropathy, and tendon tear, with examples including radiculopathy such as sciatica, tendon rupture, and impingement syndromes, chemical, toxic, or metabolic pain from direct chemical injury to tissues or nerves or metabolic imbalance, with examples including chemotherapy-induced peripheral neuropathy, alcoholic neuropathy, and uremic pruritus and pain, and thermal and electrical injury pain including burns of first to third degree with intense acute nociceptive pain and later neuropathic dysesthesia, frostbite, and electrical injury causing neuropathic and musculoskeletal pain. The lidocaine patch is also effective for treating sensory distortions and dyspesthesia including hyperpathia characterized by delayed afterdischarge pain with explosive quality, dysesthesia involving unpleasant abnormal sensations such as burning or crawling, formication presenting as crawling or itching sensation, allodynia involving pain from normally non-painful stimuli, and hyperalgesia characterized by exaggerated response to painful stimuli.

A diagnostic framework for evaluating pain presentations that can be treated with the lidocaine patch involves assessment along multiple axes including onset and time course to determine whether pain is acute versus chronic and constant versus episodic, primary mechanism suspicion to identify whether pain is nociceptive, neuropathic, nociplastic, mixed, ischemic, or inflammatory in nature, anatomical distribution to determine whether pain is focal, dermatomal, widespread, or visceral, sensory descriptors including qualities such as burning, electric, throbbing, or cramping, objective signs including redness, swelling, neurological deficits, and autonomic changes, diagnostic testing including imaging, laboratory tests, nerve conduction studies and electromyography, skin biopsy for small fiber assessment, vascular studies, and endoscopy, and response to treatments including NSAIDs, opioids, neuropathic agents, and interventional blocks which helps refine understanding of the underlying mechanism. Sensory quality characteristics provide important diagnostic information, with burning sensations typically indicating neuropathic pain, small fiber involvement, or post-radiation pain, electric or lancinating sensations suggesting neuralgias or radiculopathy, aching or dull sensations indicating nociceptive bone or muscle pain, throbbing sensations suggesting inflammatory or vascular pain, cramping or colicky sensations indicating visceral hollow-organ spasm, pressure or tightness suggesting ischemia or mass effect, tingling or prickling indicating paresthesia associated with neuropathy, shock-like sensations suggesting trigeminal neuralgia or neuroma, and freezing or icy sensations indicating cold allodynia or certain neuropathies.

Pharmaceutically acceptable excipients for use in the invention are standard in the art and also include excipients not disclosed for use in transdermal applications, such as those disclosed in Handbook of Pharmaceutical Excipients, 6th edition, edited by Raymond C Rowe et al., Published by the Pharmaceutical Press, 2009, ISBN 978 1 58212 135 2 (USA).

As used herein, the term “transdermal delivery” refers to the delivery of a compound, e.g., an active ingredient of this invention or other therapeutic agent, through one or more layers of the skin (e.g., epidermis, dermis, etc). Transdermal delivery of an active ingredient of this invention, e.g., lidocaine, may include administration of the active ingredient to the skin surface of a subject, including a human subject, so that the active ingredient passes through the skin tissue and, for example, into deeper tissue thereby providing deep tissue relief of pain.

Administration of the active ingredient or compositions of this invention includes topical administration. As used herein, the term “topical” refers to administration of a patch of this invention at the point of application. The phrase “topically applying” describes application onto one or more surfaces(s) including epithelial surfaces. Although topical administration, in contrast to transdermal administration, generally provides a local rather than a systemic effect, as used herein, unless otherwise stated or implied, the terms topical administration and transdermal administration are used interchangeably. Ideally, the substance will not reside in the skin for any extended period of time, but will penetrate into localized tissue, deep tissue and/or synovial fluids in order to provide localized, deep tissue or “joint” pain relief or any combination thereof. In one embodiment, transdermal delivery is enhanced, wherein enhancement may be through chemical or physical means.

As used herein, the term “therapeutically effective amount” refers to that amount of any active ingredient, e.g., lidocaine, which provides a therapeutic or beneficial effect for a given condition and administration regimen to a subject. The concentration of the substance is selected so as to exert its pharmaceutical effect at dosages. In certain circumstance, such dosages are low enough to avoid significant side effects to a subject. The effective amount of an active ingredient may vary with the particular site at which a patch of this invention is placed, e.g., the thickness of the skin tissue at the treatment site, the age and physical condition of the biological subject being treated, the severity of the condition, the duration of the treatment, the nature of concurrent therapy, the specific compound, composition or other active ingredient employed, the particular carrier utilized, and like factors. The effective amount of any of the active ingredients comprised in the compositions of the present invention may, for example, be the amount that results in a therapeutic or beneficial effect following its administration to a subject. The concentration of an active ingredient is selected so as to exert its pharmaceutical effect, but low enough to avoid significant side effects within the scope and sound judgment of the skilled artisan. The effective amount of the composition may vary with the particular epithelial tissue being treated, the age and physical condition of the biological subject being treated, the severity of the condition, the duration of the treatment, the nature of concurrent therapy, the specific compound, composition or other active ingredient employed, the particular carrier utilized, and like factors. As used herein, the term “therapeutically effective amount” may also be referred to herein as a “pharmaceutically effective amount”.

As used herein, the term “permeation enhancement” refers to enhancement of the percutaneous penetration of the active ingredient, allowing for a fast onset of action. As used herein, the term “permeation enhancement” may also be referred to as “transdermal enhancement” or “penetration enhancement”. In one embodiment, permeation enhancement may be performed through the use of chemical permeation enhancers.

In an exemplary embodiment, heparin is used as a chemical permeation enhancer for penetration of the active ingredient into the skin of the subject. Heparin, known for its anticoagulant activity, is surprisingly being used as a non-anticoagulant permeation enhancer in the present invention. In transdermal and topical formulations, heparin enhances the permeability of active pharmaceutical ingredients (APIs) across the skin barrier without exerting its systemic anticoagulant effects. Heparin increases the absorption of lidocaine, while allowing for more effective local anaesthesia and pain relief. In addition to its ability to hydrate the skin, heparin creates pathways for drugs to pass through, leading to improved drug delivery at the site of application.

Non-anticoagulant form of heparin is preferred to avoid the unnecessary systemic anticoagulant effects, particularly since the lidocaine patch is formulated for localized pain relief rather than systemic drug delivery in the exemplary embodiment. By optimizing the concentration of heparin and lidocaine, the formulation ensures maximum pain relief while minimizing adverse effects associated with anticoagulation.

In another embodiment, permeation enhancement may be performed through the use of physical permeation enhancers. Physical permeation enhancer techniques include magnetophoresis, iontophoresis or a battery powered electronic stimulant.

Iontophoresis, also known as Electromotive Drug Administration (EMDA), is a technique using a small electric charge to deliver a medicine, drug, active ingredient or other chemical through the skin. It may function similar to an injection without the needle, for example EMDA may be used for localized entry of a drug into the skin. In addition, EMDA may be used for concentrated application of a medication under the skin. As used herein, “iontophoresis” refers to a non-invasive method of propelling high concentrations of a charged substance, for example a medication, a drug, an active ingredient or a bioactive agent, transdermally by repulsive electromotive force using a small electrical charge applied to an iontophoretic chamber containing a similarly charged active agent and its vehicle. One or two chambers may be filled with a solution containing an active ingredient and its solvent, also called the vehicle. The positively charged chamber, called the anode, will repel a positively charged chemical, whereas the negatively charged chamber, called the cathode, will repel a negatively charged chemical into the skin.

Iontophoresis is well known for use in transdermal drug delivery. Unlike transdermal patches, this method relies on active transportation within an electric field. In the presence of an electric field electromigration and electroosmosis are the dominant forces in mass transport. These movements are measured in units of chemical flux, commonly μmol/cm2h. As described herein, iontophoresis may be used in conjunction with a patch of this invention for “permeation enhancement” of an active ingredient.

At the same time, the active ingredient must not penetrate so effectively through the skin as to be rapidly lost to the systemic circulatory systems, for example in where the lidocaine solution is not entering the blood stream. Thus, the ideal vehicle would also enhance the skin's ability to retain the pharmacologically active ingredient or, in other words, to increase skin residence times.

As used herein, the term “pain-relieving amount” refers to the amount of any of the active ingredients of this invention that results in the reduction of pain following its administration to a subject.

As used herein, the term “subject” refers to all animals including humans. Examples of patients or subjects include humans, cows, dogs, cats, goats, sheep, and pigs. As used herein, the term “subject”may also be referred to as a “patient”.

The terms “treating” or “treatment” includes, but is not limited to, the application of the patch comprising a composition comprising at least on active ingredient to the skin of a patient to prevent, reduce or inhibit the sensation of pain in the vicinity or region of the application of the patch. Further, the terms “treating” or “treatment” as used herein refer to reducing in severity and/or frequency of symptoms and/or their underlying cause of neuropathic pain.

### II. Patches for Pain Reduction

The present invention provides patches comprising a lidocaine pharmaceutical composition, wherein the lidocaine may act as an analgesic. In this way, the lidocaine can provide a regional blockage of nervous pathways that carry pain signals, thereby reducing pain suffered by a subject. In one embodiment, the composition of lidocaine includes about 1-10% by weight.

As used herein, “pharmaceutical composition” means a “therapeutically effective amount” of the active ingredient, e.g., lidocaine, together with a pharmaceutically acceptable carrier or diluent. The pharmaceutical compositions of the present invention may be a sustained or extended-release composition or an immediate release composition comprising lidocaine. An example of a pharmaceutical composition of this invention includes a therapeutically effective amount of lidocaine in a physiologically acceptable vehicle. As used herein the term “pharmaceutical composition” may also be referred to herein as a “composition”. The methods to prepare the compositions useful in the present invention are within the ordinary skill of persons in the art.

In one embodiment, a composition of this invention is administered to reduce the intensity of pain in a subject.

In one embodiment, this invention provides a patch for transdermal delivery of lidocaine for reducing pain comprising, a patch comprising a composition comprising a therapeutically effective amount of lidocaine, wherein the amount of lidocaine ranges between 1 and 10 percent by weight. As used herein, the term “lidocaine” may also be referred to herein as the “analgesic”or “local analgesic”.

The patches of this invention employ lidocaine as an active ingredient in a form capable of transdermal transport into the dermis or deeper. In one embodiment, the lidocaine can be formulated at least in part, as the free base. In another embodiment, lidocaine active ingredients can be formulated as neutralized pharmaceutically acceptable salt forms. Pharmaceutically acceptable salts include the acid addition salts, which are formed with inorganic acids such as, for example, hydrochloric, sulfuric or phosphoric acids, or such organic acids as acetic, oxalic, tartaric, mandelic, citric and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The patch delivery system of this invention comprises a composition comprising a low-dose of lidocaine. As used herein, the terms “dose” or “dosage” refer to the measured quantity of an active ingredient administered at one time. As used herein, the term “dosage” may also herein be referred to as “dose” or “amount”. In one embodiment, the composition of lidocaine comprises less than 10 percent lidocaine by weight. In one embodiment, the amount of lidocaine is between 1 percent and 10 percent. In another embodiment, the amount of lidocaine is about 1 percent. In yet another embodiment, the amount of lidocaine is about 2 percent. In yet another embodiment, the amount of lidocaine is about 3 percent. In yet another embodiment, the amount of lidocaine is about 4 percent. In still another embodiment, the amount of lidocaine is about 5 percent. In another embodiment, the amount of lidocaine is between 5 percent and 10 percent. In a further embodiment, the amount of lidocaine is 9.9 percent. In a further embodiment, the amount of lidocaine is 5.4 percent.

In yet another embodiment, the patches of the present invention can further include one or more additional compatible active ingredients which are aimed at providing the composition with another pharmaceutical effect in addition to that provided by lidocaine. “Compatible” as used herein means that the components of such a composition are capable of being combined with each other in a manner such that there is no interaction that would substantially reduce the efficacy of the composition under ordinary use conditions.

Such additional active ingredients include, but are not limited to penetration enhancers, and agents that reduce skin discomfort such as anti-inflammatory agents. In one embodiment, a combination of local anesthetics, such as are known in the art, can be comprised in a single patch.

In one embodiment, the patch of the present invention is infused with penetration enhancers or permeation enhancing agent which aid in treatment effectiveness by facilitating delivery of the lidocaine. The term “penetration enhancer” as used herein refers to an agent known to accelerate the delivery of a substance through the skin. The penetration enhancer used exemplarily in this invention includes heparin, also referred to as heparinic acid or heparinum. Other suitable penetration enhancers usable in the present invention include, but are not limited to; organic acids including but not limited to oleic acid and isosteric acid; dimethylsulfoxide (DMSO), dimethyl formamide (DMF), allantoin, urazole, N,N-dimethylacetamide (DMA), decylmethylsulfoxide (C10 MSO), polyethylene glycol monolaurate (PEGML), propylene glycol (PG), propylene glycol monolaurate (PGML), glycerol monolaurate (GML), lecithin, the 1-substituted azacycloheptan-2-ones, particularly 1-n-dodecylcyclazacycloheptan-2-one (available under the trademark AzoneRTM from Whitby Research Incorporated, Richmond, Va.), alcohols including menthol, and the like. In one embodiment, the permeation enhancement agent is menthol.

The permeation enhancer may also be a vegetable oil. Such oils include, for example, safflower oil, cottonseed oil and corn oil. Additional penetration enhancers may generally be found in Remington's Pharmaceutical Sciences, 18th, 19th or 23rd editions, published by the Mack Publishing Company of Easton, Pa, and Remington Essentials of Pharmaceutics Edited by Linda Felton, Published by Pharmaceutical Press, ISBN 978 0 885711 105 0, which is incorporated in full herein by reference.

In one embodiment, the patch of this invention further comprises a permeation enhancement agent in an amount effective to enhance treatment. In certain embodiments, the permeation enhancement agent is a component of the composition. In alternate embodiments, the permeation enhancement agent is an active ingredient. In some embodiments, the composition of this invention comprises a permeation enhancement agent. In another embodiment, the permeation enhancement agent is menthol.

As used herein, the term “enhance treatment” refers to an amount of a permeation agent needed to enhance the permeation of an active ingredient, to enhance the reduction of pain experienced by a subject or to reduce side effects including skin discomfort resulting from administration of a patch of this invention, or any combination thereof. The term “enhance treatment” may herein also be referred to as “increase effective treatment”. Enhanced treatment may result in an increase of an active ingredient permeating the skin. Alternatively, enhancement may result in a more rapid rate of an active ingredient permeating the skin then would occur without such treatment.

In one embodiment, a treatment-enhancing amount of a permeation enhancing agent can be about 0.5 percent to about 20 percent. In another embodiment, a treatment-enhancing amount of a permeation enhancer is about 0.5 percent. In another embodiment, a treatment-enhancing amount of a permeation enhancing agent can be about 5 percent to about 10 percent. In yet another embodiment, a treatment-enhancing amount of a permeation enhancing agent can be about 5 percent to about 15 percent. In yet another embodiment, a treatment-enhancing amount of a permeation enhancing agent can be about 5 percent to about 12 percent. In another embodiment, a treatment-enhancing amount of a permeation enhancing agent can be about 5 percent to about 14 percent. In yet another embodiment, a treatment-enhancing amount of a permeation enhancing agent can be about 10 percent to about 20 percent. In further embodiment, a treatment-enhancing amount of a permeation enhancing agent can be about 15 percent to about 20 percent, wherein these percentages are expressed as weight per weight of the composition comprised in the patch. In another embodiment, the permeation enhancement agent is menthol.

As used herein, reference of a percent amount of an active ingredient “by weight”, herein refers to the percent expressed as weight per weight of the composition comprised in the patch.

The patch of the present invention also can be infused with an anti-inflammatory agent to reduce skin discomfort. As used herein “inflammation” refers to a response to infection and injury in which cells involved in detoxification and repair are mobilized to the compromised site by inflammatory mediators. Thus, the body's response may include edema, vasodilation, fever and pain. The term “skin discomfort” is used herein to refer to burning, stinging, itching, tingling, loss of feeling or heightened sensitivity of the skin. “Steroidal anti-inflammatory agent”, as used herein, refer to any one of numerous compounds containing a 17-carbon 4-ring system and includes the sterols, various hormones (as anabolic steroids), and glycosides. Representative examples of steroidal anti-inflammatory drugs include, without limitation, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof. In one embodiment, a patch of this invention includes at least one anti-inflammatory agent. In another embodiment, a patch of this invention does not include an anti-inflammatory agent.

Preferably, the additional active ingredients are added in a treatment-enhancing amount. As used herein a “treatment-enhancing amount” refers to an amount that is effective to accomplish the desired effect. Typically, such an effective amount is an amount between about 0.1 up to about 10 percent as weight per weight of the composition. More typically a treatment-enhancing amount would be between about 1 to about 5 percent. In one embodiment, the patch and methods of this invention comprise lidocaine and additional active ingredients in a treatment-enhancing amount. In another embodiment, heparin enhances the delivery of the additional active ingredient.

Preferably, a treatment-enhancing amount of anti-inflammatory agent used to reduce skin discomfort is about 1 percent to about 5 percent, preferably about 1 percent to about 3 percent, and most preferably about 1 percent, wherein these percentages are expressed as weight per weight of the composition.

In other embodiment, the patch of the present invention can be used in conjunction with rehabilitation modalities, such as ultrasound, magnetophoresis, iontophoresis or a battery powered electronic stimulant. As used herein, the term “magnetophoresis” refers to the motion of dispersed magnetic particles relative to a fluid under the influence of a magnetic field. Magnetophoresis may provide enhancing drug delivery across biological barriers, including intact skin. Introduction of a drug or additional active ingredient through intact skin by the application of a direct electric current in iontophoresis may provide enhanced drug delivery when used in combination with a patch of this invention. In other embodiments, other patches described herein may be used in combination with iontophoresis. In one embodiment, iontophoresis acts as a transdermal delivery system in which a substance bearing a charge is propelled through the skin by a low electrical current. This method can be used to drive a drug across the skin barrier, as is done with pilocarpine to stimulate sweating in the sweat chloride test for cystic fibrosis. Iontophoresis can also be used in the reverse direction to draw a molecule such as glucose through the skin.

Methods utilizing electromotive enhancement treatments such as iontophoresis or magnetophoresis can provide faster relief to a subject, can increased an amount of an active ingredient penetrating into the skin or deeper tissue, can lead to an active ingredient penetrating deeper than the skin layers (epidermis, dermis), can provide longer relief from pain, can provide extended relief from pain or can provide stronger relief from pain, or any combination thereof. For example, in one embodiment, dependent on the strength of the charge used a drug may enter into skin and additionally into blood vessel found deeper under the skin.

In an additional embodiment, the patch of the present invention can be used in conjunction with rehabilitation therapies, such as heat, massage, manipulation, strength and stretching exercises, to maximize healing results with the elimination of muscle pain and spasm.

### III. Preparation Process of the Transdermal Patches:

### EXAMPLE 1

According to an embodiment a process of manufacturing transdermal patches is disclosed. The process involves weighing appropriate amounts of pharmaceutically active ingredients, inactive ingredients and adhesives weighed in a container. In an exemplary embodiment, lidocaine is the active ingredients in the composition. In one embodiment, the composition of lidocaine comprises less than 10 percent lidocaine by weight. In one embodiment, the amount of lidocaine is between 1 percent and 10 percent. In another embodiment, the amount of lidocaine is about 1 percent. In yet another embodiment, the amount of lidocaine is about 2 percent. In yet another embodiment, the amount of lidocaine is about 3 percent. In yet another embodiment, the amount of lidocaine is about 4 percent. In still another embodiment, the amount of lidocaine is about 5 percent. In another embodiment, the amount of lidocaine is between 5 percent and 10 percent. In a further embodiment, the amount of lidocaine is 9.9 percent.

In the exemplary embodiment, heparin is a permeation enhancer in the composition. In one embodiment, the composition of the permeation enhancing agent can be up to about 50 percent. In another embodiment, the composition of the permeation enhancing agent can be about 1 percent to about 20 percent. In yet another embodiment, the composition of the permeation enhancing agent can be about 1 percent to about 10 percent. In yet another embodiment, the composition of the permeation enhancing agent can be about 1 percent to about 15 percent. In further embodiment, the composition of the permeation enhancing agent can be about 5 percent to about 10 percent. In another embodiment, the composition of the permeation enhancing agent can be about 5 percent to about 12 percent, wherein these percentages are expressed as weight per weight of the composition comprised in the patch. In yet another embodiment, the composition of the permeation enhancing agent can be about 5 percent to about 14 percent. In yet another embodiment, the composition of the permeation enhancing agent can be about 3 percent to about 20 percent. In yet another embodiment, the composition of the permeation enhancing agent can be about 5 percent to about 15 percent. In further embodiment, the composition of the permeation enhancing agent can be about 5 percent to about 20 percent. In another embodiment, the composition of the permeation enhancing agent can be about 5 percent to about 17 percent, wherein these percentages are expressed as weight per weight of the composition comprised in the patch. In another embodiment, the composition of the permeation enhancing agent is about 50 percent weight per weight. In the exemplary embodiment, the adhesive is styrene-isoprene-styrene block copolymer.

Upon weighing, the ingredients are dissolved or suspended in the adhesive solution and then the solution is mixed until homogeneous. A sheet of release liner is placed onto a patch coater such as Warner Mathis coater. The solution is poured on the release liner and a thin film of solution is coated on the release liner. The solution is dried by placing it in an oven which is preset at temperatures of 60° C. and drying time of 10 minutes to evaporate the solvent. After drying, the dried film is laminated with a sheet of backing layer. Finally, the laminate is cut with die cutter into desired sizes.

### EXAMPLE 2

Adhesive: Styrene-isoprene-styrene block copolymer (“Kraton D1161”): 20 mass %, Polyisobutylene (“Himol 6H”): 10 mass % and Terpene resin (“YS resin 1150N”): 20 mass %

Softener: Liquid paraffin (“Hicall”): 45.1 mass %

Dissolving Agents: Isostearic acid: 0.9 mass % and Dipropylene glycol: 0.2 mass %

Local anaesthesia: Lidocaine: 0.5 mass %

Permeation Enhancer: Heparin: 5 mass % as a non-anticoagulant

Regulator: Light anhydrous silicic acid (“Sylysia 350”): 0.5 mass %

Additive Agent: Dibutylhydroxytoluene (BHT): 0.3 mass %

The method of manufacturing transdermal patch using aforementioned composition is disclosed. The method involves placing one or more adhesive agents in a dissolution mixer and dissolving the adhesive agents under heating at 150° C. Then a solution separately prepared by mixing the lidocaine, permeation enhancing agents such as heparin, and a dissolving agents is added to the adhesive solution followed by dissolution at 80° C. Further, the mixture is mixed under heating at 140° C. until the mixture became homogeneous, thereby obtaining a plaster solution.

The plaster solution was applied to a polyester film treated with silicon so that the plaster weight is 140 g/m2. A polyester knitted fabric, of which strength of 50% stretched to longitudinal direction is 1600 g/50 mm, is pasted to the film and cooled. The resultant is then cut into a rectangle (about 14 cm×10 cm).

### EXAMPLE 3

Adhesive: Styrene-isoprene-styrene block copolymer (“Kraton D1161”): 20 mass %, Polyisobutylene (“Himol 6H”): 10 mass % and Terpene resin (“YS resin 1150N”): 20 mass %

Softener: Liquid paraffin (“Hicall”): 35.4 mass %

Dissolving Agents: Isostearic acid: 1.8 mass % and Dipropylene glycol: 0.5 mass %

Local anaesthesia: Lidocaine: 1.5 mass %

Permeation Enhancer: Heparin: 10 mass % as a non-anticoagulant

Regulator: Light anhydrous silicic acid (“Sylysia 350”): 0.5 mass %

Additive Agent: Dibutylhydroxytoluene (BHT): 0.3 mass %

The method of manufacturing transdermal patch using aforementioned composition is disclosed. The method involves placing one or more adhesive agents in a dissolution mixer and dissolving the adhesive agents under heating at 150° C. Then, a solution separately prepared by mixing the lidocaine, permeation enhancing agents such as heparin, and a dissolving agents is added to the adhesive solution followed by dissolution at 80° C. Further, the mixture is mixed under heating at 140° C. until the mixture became homogeneous, thereby obtaining a plaster solution.

The plaster solution is applied to a polyester film treated with silicon so that the plaster weight is 140 g/m2. A polyester non-woven fabric, of which strength of 50% stretched to longitudinal direction is 1000 g/50 mm, is pasted to the film and cooled. The resultant is then cut into a rectangle (about 14 cm×10 cm).

### EXAMPLE 4

Adhesive: Styrene-isoprene-styrene block copolymer (“Kraton D1161”): 20 mass %, Polyisobutylene (“Himol 6H”): 10 mass % and Terpene resin (“YS resin 1150N”): 20 mass %.

Softener: Liquid paraffin (“Hicall”): 29.9 mass %

Dissolving Agents: Isostearic acid: 1.8 mass % and Dipropylene glycol: 0.5 mass %

Local anaesthesia: Lidocaine: 2 mass %

Permeation Enhancer: Heparin: 15 mass % as a non-anticoagulant

Regulator: Light anhydrous silicic acid (“Sylysia 350”): 0.5 mass %

Additive Agent: Dibutylhydroxytoluene (BHT): 0.3 mass %

The method of manufacturing transdermal patch using aforementioned composition is disclosed. The method involves placing one or more adhesive agents in a dissolution mixer and dissolving the adhesive agents under heating at 150° C. Then a solution separately prepared by mixing the lidocaine, permeation enhancing agents such as heparin, and a dissolving agents is added to the adhesive solution followed by dissolution at 80° C. Further, the mixture is mixed under heating at 140° C. until the mixture became homogeneous, thereby obtaining a plaster solution.

The plaster solution is applied to a polyester film treated with silicon so that the plaster weight is 160 g/m2. A polyester non-woven fabric, of which strength of 50% stretched to longitudinal direction is 500 g/50 mm, is pasted to the film and cooled. The resultant is then cut into a rectangle (about 14 cm×10 cm).

### EXAMPLE 4

Adhesive: Styrene-isoprene-styrene block copolymer (“Kraton D1161”): 20 mass %, Polyisobutylene (“Himol 6H”): 10 mass % and Terpene resin (“YS resin 1150N”): 20 mass %.

Softener: Liquid paraffin (“Hicall”): 18 mass %

Dissolving Agents: Isostearic acid: 2.5 mass % and dipropylene glycol: 1.5 mass %

Local anaesthesia: Lidocaine: 7 mass %

Permeation Enhancer: Heparin: 20 mass % as a non-anticoagulant

Regulator: Light anhydrous silicic acid (“Sylysia 350”): 0.7 mass %

Additive Agent: Dibutylhydroxytoluene (BHT): 0.3 mass %

The method of manufacturing transdermal patch using aforementioned composition is disclosed. The method involves placing one or more adhesive agents in a dissolution mixer and dissolving the adhesive agents under heating at 150° C. Then, a solution separately prepared by mixing the lidocaine, permeation enhancing agents such as heparin, and a dissolving agents is added to the adhesive solution followed by dissolution at 80° C. Further, the mixture is mixed under heating at 140° C. until the mixture became homogeneous, thereby obtaining a plaster solution.

The plaster solution is applied to a polyester film treated with silicon so that the plaster weight is 160 g/m2. A polyester non-woven fabric, of which strength of 50% stretched to longitudinal direction is 500 g/50 mm, is pasted to the film and cooled. The resultant is then cut into a rectangle (about 14 cm×10 cm). Other manufacturing processes of the patch can be found as described in Dermal Drug Delivery, From Innovation to Production, (edited by Tapash K. Ghosh, 2020), hereby incorporated in full herein by reference.

Heparin acts as an effective permeation enhancer for lidocaine, while significantly improving its ability to penetrate the skin barrier. Heparin temporarily disrupts the lipid bilayers in this layer, and creates microchannels that allow for increased diffusion of lidocaine molecules. This enhanced permeation leads to higher local concentrations of lidocaine in the target tissues, thereby potentially improving the analgesic effect without increasing the overall dose of the drug.

When used as a permeation enhancer in lidocaine patches, heparin can help extend the duration of the analgesic effect. This is achieved through two mechanisms: first, by enhancing the initial penetration of lidocaine, and second, by potentially forming a depot effect in the skin. The interaction between heparin and lidocaine may create a reservoir of the drug in the upper layers of the skin, allowing for a more sustained release over time. This prolonged duration can lead to improved pain management with less frequent patch applications, while enhancing patient compliance and quality of life.

While heparin enhances the local penetration of lidocaine, it does not necessarily increase systemic absorption of the drug. This is advantageous as it allows for effective local anaesthesia without significantly elevating blood levels of lidocaine. By keeping the drug localized to the site of application, the risk of systemic side effects is minimized. The localized effect is beneficial for patients who may be sensitive to systemic lidocaine or those with conditions that contraindicate high systemic levels of local anesthetics.

By enhancing the permeation of lidocaine, the use of heparin as an excipient may allow for a reduction in the overall lidocaine content in the patch. The lower drug load can have several benefits: it may reduce the cost of production, minimize the risk of lidocaine-related side effects, and potentially improve the patch's safety profile. The ability to achieve therapeutic effects with lower drug concentrations is advantageous in long-term pain management scenarios.

In various exemplary embodiments, heparin serves as an enhancer in lidocaine patches, facilitating enhanced release of lidocaine from the patch and increasing its bioavailability in the body. When heparin is combined with lidocaine, it modifies the release kinetics, enabling more lidocaine to be released from the patch surface and absorbed through the skin. This enhancement occurs even at lidocaine doses that are at the same or lower levels than those typically used, thereby potentially improving therapeutic outcomes without increasing the lidocaine dose. This implies that with heparin's inclusion, the effectiveness of the patch in delivering lidocaine to the target area is improved, achieving more substantial pain relief while minimizing the need for higher doses of lidocaine, which can reduce the risk of adverse effects associated with larger doses.

Heparin, when used in non-anticoagulant doses typical for topical applications, generally has a safety profile. It is a naturally occurring substance in the human body, which contributes to its biocompatibility. When used topically in a lidocaine patch, the risk of systemic effects related to heparin's anticoagulant properties is minimal. This makes it a safe choice for enhancing drug permeation, especially compared to some synthetic chemical enhancers that may have more significant side effects or toxicity concerns.

### IV. Patch Administration for Pain Reduction

Patches of the present invention have been described above. The patches can be administered at or adjacent to a site of pain to provide relief. In one embodiment, the pain is neuropathic pain, wherein administration of a patch of this invention reduces the neuropathic pain felt by a subject. In another embodiment, the pain is osteoarthritis pain, wherein administration of a patch of this invention reduces the osteoarthritic pain felt by a subject. In yet another embodiment, the pain is back pain, wherein administration of a patch of this invention reduces the back pain felt by a subject. In still another embodiment, the pain is a result of bone degeneration, wherein administration of a patch of this invention reduces the bone degeneration pain felt by a subject. In a further embodiment, the pain is associated with fibromyalgia, wherein administration of a patch of this invention reduces the pain associated with fibromyalgia felt by a subject. In another embodiment, the pain is associated with muscle strain, wherein administration of a patch of this invention reduces the muscle strain pain felt by a subject. In yet another embodiment, the pain is associated with muscle sprain, wherein administration of a patch of this invention reduces the muscle sprain pain felt by a subject. In still another embodiment, the pain is that associated with carpal tunnel syndrome pain, wherein administration of a patch of this invention reduces the carpal tunnel syndrome pain felt by a subject. In a further embodiment, the pain is that associated with any combination of diseases or disorders able to be relieved by local and/or deep tissue pain relief, wherein administration of a patch of this invention reduces the pain felt by a subject.

In one embodiment, a method for reducing pain in a subject comprises applying on a skin surface of the subject, at or near the site of pain, a patch comprising a composition of lidocaine and heparin, wherein the application provides for transdermal delivery of an amount of lidocaine sufficient to reduce neuropathic pain in the subject. In one aspect, lidocaine is an active ingredient while heparin acts a permeation enhancer having non-anticoagulant properties. The methods of this invention may reduce pain resulting from different diseases, disorders or condition including neuropathic pain, osteoarthritis pain, back pain, degenerative bone pain, pain associated with carpal tunnel syndrome, pain associated with fibromyalgia, pain associated with muscle strain or pain associated with muscle sprain or any combination thereof.

As used herein, the term “administration” refers to applying, e.g., adhering a patch comprising a lidocaine formulation on a skin surface of a subject.

In one embodiment, administration of a patch provides immediate or nearly immediate relief, e.g., reduction of pain. In another embodiment, administration of a patch provides long term relief, e.g., reduction of pain. In yet another embodiment, administration of a patch provides both immediate or nearly immediate and long-term relief, e.g., reduction of pain.

The patches described herein can be administered at or adjacent to the sites of pain to provide relief. The patches can be administered once a day, for example, for fast, long term pain relief, e.g., pain relief is starts relatively quickly and is maintained over an extended period of time. As used herein the terms “reduction of pain” and “pain relief” are interchangeable with all the same meanings. In one embodiment, application of a patch may reduce the pain suffered by a subject completely or almost completely. In another embodiment, application of a patch may reduce the pain suffered by a subject by about 50 to almost 100 percent. In yet another embodiment, application of a patch may reduce the pain suffered by a subject by about 50 to 90 percent. In still another embodiment, application of a patch may reduce the pain suffered by a subject by about 80 to 90 percent. In a further embodiment, application of a patch may reduce the pain suffered by a subject by about 70 to 80 percent. In one embodiment, application of a patch may reduce the pain suffered by a subject by about 50 to 70 percent. In still another embodiment, application of a patch may reduce the pain suffered by a subject by less than 50 percent.

In one embodiment a patch of this invention is applied for between about 8 to 12 hours. In one embodiment a patch is applied for about 8 hours. In another embodiment a patch is applied for about 9 hours. In yet another embodiment, a patch is applied for about 10 hours. In still another embodiment, a patch is applied for about 11 hours. In a further embodiment, a patch is applied for about 12 hours. In still another embodiment, a patch is applied for about 1 day to about 30 days.

A patch of the present invention is applied on the skin surface at a site or adjacent to a painful region. In some embodiments, multiple patches may be applied at the same time. Patches may be applied in the same region, an adjacent region or regions distal from one another. Fresh patches may be reapplied after a 24-hour period counted from the time the previous patch was administered.

In some embodiments, the patch is applied to the painful skin and subcutaneous structures in order to effect pain relief while avoiding the side effects associated with systemic delivery. Pain relief is obtained within minutes to hours and lasts for periods of approximately three to six hours to 24 hours. The patches are applied such that the dosage is sufficient to provide an effective dose in the painful area or immediately adjacent areas, to ameliorate or eliminate pain and other unpleasant sensations such as itching. In one embodiment, pain is reduced for at least 12 hours. In another embodiment, pain is reduced for at least 24 hours. In yet another embodiment, pain is reduced for more than 24 hours.

The appropriate dosages for pain treatment by way of patches of the present invention are determined by a variety of factors. The rate at which the active components are absorbed is a function of skin permeability. Skin permeability varies between different sites on a patient's body and depends on the thickness of the stratum corneum. The stratum corneum is the outer-most layer of skin and is the main source of penetration and permeation resistance for dermally administered drugs. For example, the permeability, in general, increases in order from planter foot arch, lateral ankle, palm, ventral forearm, dorsal forearm, back, chest, thigh, abdomen, scalp, axilla, forehead, and scrotum; see R. C. Wester. & H. I. Maibach, Regional variation in Percutaneous Absorption, in Percutaneous Absorption, Mechanism, Methodology, Drug Delivery 111-119 (R. L. Bronaugh & H. I. Maibach eds., 2nd ed. 1989), hereby expressly incorporated in full herein by reference.

The delivery rate of an active ingredient from a patch, e.g., lidocaine, of the present invention that is required for proper pain relief is determined by a variety of factors. One important factor regarding delivery rate is the surface areas of the active ingredients in contact with a patient's skin. In general, the larger the contact surface area, the higher the rate of delivery. Different delivery rates of an active ingredient may be needed depending on the severity of pain felt. The surface areas of components can adjust to provide the desired delivery rate of an active ingredient to a patient.

In addition, delivery rate may be enhanced as describe above using chemical enhancement agents, for example heparin, menthol and/or physical enhancement methodologies, for example iontophoresis, a battery powered electronic stimulant or magnetophoresis. In one embodiment, methods of this invention for reducing neuropathic pain include treatment enhancing amounts of a permeation agent. In one embodiment, methods of this invention for reducing neuropathic pain include treatment enhancing methodologies including iontophoresis, a battery powered electronic stimulant or magnetophoresis.

Methods of this invention include transdermal administration of active ingredients, e.g., lidocaine.

In one embodiment, methods of this invention for reducing pain in a subject comprise applying on a skin surface of the subject, at or near the site of pain a patch comprising compositions comprising lidocaine at 0.5 percent to 10 percent by weight. In another embodiment, methods of this invention use lidocaine at between 1 and 9 percent by weight. In yet another embodiment, methods of this invention use lidocaine at 1 percent by weight. In still another embodiment, methods of this invention use lidocaine at about 5 percent by weight. In a further embodiment, methods of this invention use lidocaine at about 6 percent by weight. In one embodiment, methods of this invention use lidocaine at about 7 percent by weight. In another embodiment, methods of this invention use lidocaine at about 8 percent by weight. In yet another embodiment, methods of this invention use lidocaine at about 9 percent by weight. In further embodiment, methods of this invention use lidocaine at between 5 and 9 percent by weight. In another embodiment, methods of this invention use lidocaine at between 5 and 10 percent by weight. In another embodiment, methods of the invention use lidocaine at about 5.4 percent.

In one embodiment the composition of this invention comprises local anesthetic and drugs not traditionally associated with local anesthetic properties but which have a local anesthetic effect. Non limiting examples of such drugs include for example, non-narcotic analgesics, such as, acetylsalicylic acid, ketoprofen, piroxicam, diclofenac, indomethacin, ketorolac, rofecoxib, and celecoxib, and pharmaceutically acceptable salts thereof, or mixtures thereof.

As used herein the term “drug” refers to a substance used in the diagnosis, treatment, or prevention of a disease or medical condition or an active component of a medication. Of course, the term “drug” encompasses local anesthetics and/or analgesics. In one embodiment, a drug of this invention is lidocaine.

A first step in any treatment method of this invention may be selection of an appropriate patch for reduction of the pain suffered by a subject. Patches may be cut to the size and shape need to administer an appropriate dose and/or cover a particular surface of skin. For instance, an appropriate patch may be perforated to provide flexibility.

Following this, a patch may be applied to the skin surface for an effective time period. For effective use, a patch should be in contact with a surface of the skin and remain in place for the duration of the treatment. In order that a patch remains in place, an adhesive may be comprised as part of a patch. Alternatively, an adhesive, such as an adhesive strip or tape may be used to hold the patch in place. For example, a patch may be adhered to a patient's back through the use of a drug-in-adhesive patch. In an alternative example, a patch may both included an adhesive and be perforated to allow the patch to stretch, wherein a patch may be wrapped fully or partially around a subject's body appendage, e.g., a leg, arm, finger or neck.

The selected patch may comprise a composition comprising permeation enhancers. In an exemplary embodiment, the permeation enhancers are chemical permeation enhancers such as heparin. Alternatively, physical methodologies such as iontophoresis, a battery powered electronic stimulant or magnetophoresis may be employed to enhance the permeation of at least one active ingredient. Technique using a small electric charge to deliver active ingredient through the skin, e.g., iontophoresis, may enhance permeation of at least one active ingredient. Iontophoresis may be imagined to be an injection without the needle. The process is a non-invasive method of propelling high concentrations of a charged substance, e.g., lidocaine, transdermally by repulsive electromotive force using a small electrical charge applied to an iontophoretic chamber containing a similarly charged active agent and its vehicle.”

Following the recommended time period of application, the patch may be removed from the skin surface. Relief from pain may continue even though the patch has been removed. Following a period of 24 from the initial administration, this cycle of use may be employed by a subject. This may be significant for sufferers of chronic pain.

In one embodiment, the term “a” or “one” or “an” refers to at least one. As used in the specification and claims, the forms “a,” “an” and “the” include singular as well as plural references unless the context clearly dictates otherwise.

In one embodiment the phrase “two or more” may be of any denomination, which will suit a particular purpose. In one embodiment, “about” may comprise a deviance from the indicated term of +1 %, or in some embodiments, −1 %, or in some embodiments, ±2.5 %, or in some embodiments, ±5 %, or in some embodiments, ±7.5 %, or in some embodiments, ±10 %, or in some embodiments, ±15 %, or in some embodiments, ±20 %, or in some embodiments, ±25 %.

While certain features of the invention have been described herein, many modifications, substitutions, changes, and equivalents will now occur to those of ordinary skill in the art. It is, therefore, to be understood that the appended claims are intended to cover all such modifications and changes as fall within the true spirit of the invention.

1. A transdermal patch for delivering lidocaine comprising:
a backing layer;
an adhesive layer;
a composition comprising:
lidocaine in an amount ranging from about 1% to about 10% by weight of the composition; and
heparin as a non-anticoagulant permeation enhancer in an amount ranging from about 1% to about 50% by weight of the composition; wherein the heparin enhances permeation of the lidocaine through skin without exerting systemic anticoagulant effects.

2. The transdermal patch of claim 1, wherein the heparin is present in an amount ranging from about 5% to about 20% by weight of the composition.

3. The transdermal patch of claim 1, wherein the heparin is present in an amount ranging from about 10% to about 30% by weight of the composition.

4. The transdermal patch of claim 1, wherein the heparin is present in an amount ranging from about 20% to about 50% by weight of the composition.

5. The transdermal patch of claim 1, wherein the lidocaine is present in an amount ranging from about 5% to about 10% by weight, and the heparin is present in an amount ranging from about 10% to about 30% by weight.

6. The transdermal patch of claim 1, wherein the adhesive layer comprises an adhesive selected from the group consisting of styrene-isoprene-styrene block copolymer, polyisobutylene-based adhesives, silicone-based adhesives, acrylic-based adhesives, polyacrylates, polydimethylsiloxane, hydrogels, and combinations thereof.

7. The transdermal patch of claim 1, further comprising one or more pharmaceutically acceptable excipients selected from the group consisting of liquid paraffin, isostearic acid, dipropylene glycol, light anhydrous silicic acid, dibutylhydroxytoluene, and combinations thereof.

8. The transdermal patch of claim 1, wherein the patch is perforated and stretchable to allow wrapping around a body appendage selected from the group consisting of knee, ankle, leg, elbow, wrist, finger, arm, and neck.

9. The transdermal patch of claim 1, wherein the heparin temporarily disrupts lipid bilayers in the stratum corneum to create microchannels for increased diffusion of lidocaine molecules, and wherein the heparin forms a depot effect in the skin allowing for sustained release of lidocaine over a period ranging from about 8 hours to about 24 hours.

10. A method for reducing pain in a subject comprising:
applying to a skin surface of the subject, at or near a site of pain, a transdermal patch comprising:
lidocaine in an amount ranging from about 1% to about 10% by weight; wherein the application provides transdermal delivery of an amount of lidocaine sufficient to reduce pain in the subject, and wherein the pain is selected from the group consisting of neuropathic pain, inflammatory pain, ischemic pain, post-surgical pain, nociplastic pain, mechanical pain, compressive pain, arthritis, back pain and combinations thereof.

11. The method of claim 10, wherein the neuropathic pain is selected from the group consisting of diabetic neuropathy, postherpetic neuralgia, carpal tunnel syndrome, chemotherapy-induced neuropathy, trigeminal neuralgia, peripheral neuropathic pain, central neuropathic pain, multiple sclerosis-related pain, post-stroke pain, spinal cord injury pain, phantom limb pain, complex regional pain syndrome Type I, complex regional pain syndrome Type II, sciatica, radiculopathy, nerve compression pain, HIV sensory neuropathy, Guillain-Barré syndrome pain, and combinations thereof.

12. The method of claim 10, wherein the inflammatory pain is selected from the group consisting of:
acute inflammatory pain including cellulitis, abscess, septic arthritis, osteomyelitis, traumatic sprains, traumatic strains, and post-operative inflammation;
chronic inflammatory pain including rheumatoid arthritis, psoriatic arthritis, ankylosing spondylitis, Crohn's disease, ulcerative colitis, chronic prostatitis, chronic pelvic inflammatory pain, and vasculitic neuropathy;
inflammatory visceral pain including pancreatitis, appendicitis, cholecystitis, and peritonitis;
inflammatory bone pain including osteomyelitis and osteitis condensans;
neuroinflammatory pain including cytokine-mediated neuropathic pain from HIV, autoimmune diseases, or CAR-T syndrome;
musculoskeletal inflammatory pain including gout, tendonitis, bursitis, and polymyalgia rheumatica;
vasculitis-related pain including giant cell arteritis and cutaneous vasculitis; and combinations thereof.

13. The method of claim 10, wherein the ischemic pain is selected from the group consisting of cardiac ischemic pain including angina pectoris and myocardial infarction, limb ischemia including intermittent claudication and acute limb ischemia, visceral ischemia including mesenteric ischemia, renal ischemia, and intestinal ischemia, ischemic neuropathic pain, sickle cell vaso-occlusive pain, ischemic fasciitis, compartment syndrome, and combinations thereof.

14. The method of claim 10, wherein the post-surgical pain is selected from the group consisting of acute postoperative pain, subacute postoperative pain, chronic post-surgical pain persisting beyond three months, post-mastectomy pain syndrome, post-hernia repair ilioinguinal neuralgia, post-amputation pain, phantom limb pain, neuroma pain, post-thoracotomy pain syndrome, chronic intercostal neuralgia, mesh-related pain, visceral adhesion pain, ischemic flap pain, graft pain, device-related pain, and combinations thereof.

15. The method of claim 10, wherein the nociplastic pain is characterized by altered central nociceptive processing involving central sensitization, dysfunctional descending inhibition, or augmented facilitatory pathways without clear ongoing peripheral tissue damage or nerve lesion, and wherein the nociplastic pain is selected from the group consisting of fibromyalgia, chronic widespread pain syndrome, irritable bowel syndrome, temporomandibular disorders, chronic tension-type headache, chronic pelvic pain syndrome, interstitial cystitis, bladder pain syndrome, non-cardiac chest pain, post-COVID chronic pain syndrome, and combinations thereof.

16. The method of claim 10, wherein the pain is selected from the group consisting of:
back pain caused by herniated disc, bulging disc, spondylolisthesis, degenerative disc disease, spinal stenosis, ankylosing spondylitis, sacroiliitis, sciatica, or nerve entrapment, wherein said pain is neuropathic or nociceptive or the combination of both;
arthritis pain including osteoarthritis, rheumatoid arthritis, psoriatic arthritis, ankylosing spondylitis, gout, lupus arthritis, juvenile idiopathic arthritis, and reactive arthritis;
muscle pain including muscle strain pain, muscle sprain pain, and muscle contusion pain;
bone pain associated with bone degeneration, bone breaks, bone fractures, bone contusions, or osteoporosis;
cancer-related pain including tumor-related pain, bone pain, chemotherapy-induced pain, radiation-induced pain, and nerve compression pain;
headache pain including tension headache, migraine, and cluster headache; and combinations thereof.

17. The method of claim 10, wherein the transdermal patch further comprising heparin in an amount ranging from about 5% to about 20% by weight, and wherein the transdermal patch is applied for a duration ranging from about 8 hours to about 24 hours.

18. The method of claim 10, wherein the pain is reduced by at least 50% following application of the transdermal patch, and wherein pain relief begins within minutes to hours of application and lasts for at least 12 hours.

19. The method of claim 10, wherein the method further comprises enhancing permeation using a technique selected from the group consisting of iontophoresis, magnetophoresis, battery-powered electronic stimulation, and combinations thereof, or wherein the method further comprises rehabilitation therapy selected from the group consisting of heat therapy, massage, manipulation, strength exercises, stretching exercises, and combinations thereof.

20. The method of claim 10, wherein the transdermal patch is wrapped around a body appendage selected from the group consisting of knee, ankle, leg, elbow, wrist, finger, arm, and neck, or wherein multiple transdermal patches are applied simultaneously at different sites of pain, and wherein the heparin enhances permeation of the lidocaine by at least 30% compared to a patch without heparin.
